(19) United States
(12) Patent Application Publication
(10) Pub. No.: US 2017/0219523 A1
(43) Pub. Date: Aug. 3, 2017

(54) EXTRACTION AND CONCENTRATION DEVICE

(71) Applicant: UNIVERSITY OF TASMANIA, Sandy Bay (AU)

(72) Inventors: Aliaa Ibrahim Gaber SHALLAN, Cairo (EG); Rosanne Marieke GUIJT, Howrah (AU); Michael Charles BREADMORE, Margate (AU)

(21) Appl. No.: 15/515,249

(22) PCT Filed: Sep. 30, 2015
(86) PCT No.: PCT/AU2015/050589

§ 371 Date: Mar. 29, 2017

(30) Foreign Application Priority Data
Oct. 1, 2014 (AU) 2014903920

(51) Int. Cl. G01N 27/447; B01D 17/06; G01N 15/02; G01N 33/487; B01L 3/00; G01N 1/40; B01D 63/00; B01D 71/50

(52) U.S. Cl. CPC G01N 27/44791; B01D 63/005; B01D 17/06; B01D 71/50; G01N 33/48721; B01L 3/50273; B01L 3/502753; G01N 1/40; G01N 15/0266; B01L 2400/0421; B01L 2300/0864; G01N 2001/4038

(57) ABSTRACT

A device for extracting and concentrating a target analyte including a sample channel that receives the sample, a separation channel, a waste channel, a first junction between the sample channel and the separation channel, and, a second junction between the separation channel and the waste channel. The first junction selectively transports a first group of analytes, including target analytes, from the sample channel to the separation channel in accordance with a size of a first free transport region of the first junction. The second junction selectively transports a second group of analytes from the separation channel to the waste channel in accordance with a size of a second free transport region of the second junction, the second group being a subset of the first group, so as to concentrate a number of the target analytes in the separation channel.

### BACKGROUND OF THE INVENTION

The present invention relates to a device for at least partially extracting and concentrating a target analyte from a fluidic sample containing a plurality of analytes.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

In the interests of improving global health, it is desirable to access diagnostic and/or analytical devices in order to facilitate fast and accurate analysis directly from biological samples. Such analyses, qualitative and quantitative, are particularly beneficial in a wide range of fields such as therapeutic drug monitoring, forensics, and diagnostics. It can also be extended to cover applications in veterinary medicine, food safety, and environmental analysis.

Traditional analytical systems for biological samples include liquid chromatography tandem mass spectrometry (LC-MS/MS) and immunoassays. Whilst LC-MS/MS has increased sensitivity and specificity compared with immunoassays, the technique is more involved, takes longer and requires expensive, non-portable equipment which must be operated by professionally trained personnel. Immunoassays are less expensive and can be portable, however they tend to suffer from specificity and sensitivity issues.

In recent times, consumer diagnostic devices have become popular and readily available in the market, such as glucose meters and pregnancy tests. Other examples for direct analysis from biological fluids include diagnostic paper microfluidics (see, A. W. Martinez, S. T. Phillips, G. M. Whitesides and E. Carrilho, Analytical Chemistry, 2009, 82, 3-10) and electrophoretic devices for glutathione analysis (see, Z. Long, D. Liu, N. Ye, J. Qin and B. Lin, Clinical Chemistry, 2007, 53, 117-123). Additionally, a microfluidic sample-in/answer-out device previously proposed for small molecules is the Medimate, which is used for quantifying the mood stabilizer lithium (see, E. X. Vrouwe, R. Luttge and A. van den Berg, Electrophoresis, 2004, 25, 1660-1667; E. X. Vrouwe, R. Luttge, W. Olthuis and A. van den Berg, Electrophoresis, 2005, 26, 3032-3042; E. X. Vrouwe, R. Luttge, I. Vermes and A. van den Berg, Clin. Chem, 2007, 53, 117-123).

However, such devices are typically limited to analyse certain analytes. The presence of large hydrophobic molecules, like proteins and lipids, in biological samples interferes with analysis. In the Medimate, whilst protein adsorption is reduced using a surface coating, it is not eliminated, thus precluding the device from wider applicability to measure other analytes.

Thus, whilst consumer diagnostic devices are becoming more prevalent, existing devices suffer from one or more drawbacks, including low sensitivity and/or resolution, lengthy and/or complex sample preparation, high and/or complex instrumentation and training requirements, lengthy processes, high manufacturing costs, and the like.

### SUMMARY OF THE PRESENT INVENTION

The present invention seeks to ameliorate one or more of the problems associated with the prior art and/or provide a workable alternative.

In one broad form the present invention seeks to provide a device for at least partially extracting and concentrating a target analyte from a fluidic sample containing a plurality of analytes, the device including:
a) a sample channel that receives the sample;
b) a separation channel;
c) a waste channel;
d) a first junction between the sample channel and the separation channel, wherein the first junction selectively transports a first group of analytes from the sample channel to the separation channel in accordance with a size of a first free transport region of the first junction, the first group including at least some target analytes and being a subset of the plurality of analytes in the sample; and,
e) a second junction between the separation channel and the waste channel, wherein the second junction selectively transports a second group of analytes from the separation channel to the waste channel in accordance with a size of a second free transport region of the second junction, the second group being a subset of the first group, so as to concentrate a number of the target analytes in the separation channel.

Typically the first junction includes at least one first junction channel extending between the sample channel and the separation channel, and wherein the size of the first free transport region is at least partially dependent upon at least one of a size of the at least one first junction channel and a degree of an electric double layer overlap within the at least one first junction channel.

Typically the second junction includes at least one second junction channel extending between the separation channel and the waste channel, and wherein the size of the second free transport region is at least partially dependent upon at least one of a size of the at least one second channel and a degree of an electric double layer overlap within the at least one second junction channel.

Typically the selective transport in the first junction is in accordance with at least one of a charge and size of each of the analytes in the first group.

Typically the selective transport in the second junction is in accordance with at least one of a charge and size of each of the analytes in the second group.

Typically the device includes at least one first electrode in the sample channel, and at least one second electrode in the waste channel, to thereby apply a first electric potential across the first and second junctions so as to selectively transport analytes through the first and second junctions.

Typically the first electric potential is applied to thereby sharpen the concentrated target analytes within a region of the separation channel.

Typically the device includes third and fourth electrodes in the separation channel to thereby apply a second electric potential along the separation channel so as to selectively transport analytes from the first group of analytes within the separation channel.

Typically the electric potential in the separation channel is used to cause target analytes to migrate at a speed based on at least one of a size, charge ratio, and electrophoretic mobility of analytes.

Typically the device includes a detector that detects a concentration of the target analytes within the separation channel in use.

Typically at least one of the first and the second junctions includes at least one of:
a) a plurality of channels;
b) a single channel having an elongate cross sectional area;
c) a hydrogel; and,
d) a membrane.

Typically the first junction and the second junction are offset along a length of the separation channel.

Typically at least one of the sample channel and the waste channel is at least one of:
a) tapered toward the separation channel;
b) substantially “V” shaped; and,
c) substantially “U” shaped.

Typically the sample channel includes two sample channel arms, each arm having a respective first electrode proximate a first end and the first junction being provided proximate a second opposing end.

Typically the waste channel includes two waste channel arms, each arm having a respective second electrode proximate a first end and the first junction being provided proximate a second opposing end.

Typically the selective transport in the second junction is at least partially controlled in accordance with at least one of electrophoretic mobility, electroosmotic flow (EOF), and ion concentration polarization.

Typically the device includes a number of second junctions spaced apart along the separation channel, and wherein each second junction is for removing respective analytes thereby allowing a number of different target analytes to be extracted and concentrated within the separation channel.

In another broad form the present invention seeks to provide apparatus for at least partially extracting and concentrating a number of target analytes from a fluidic sample containing a plurality of analytes, the apparatus including a number of devices according to a broad form of the invention, each device being adapted to extract and concentrate a respective analyte and wherein the waste channel of an upstream device is at least one of in fluid communication with and forms part of a sample channel of a downstream device.

In another broad form the present invention seeks to provide a method of at least partially extracting and concentrating a target analyte from a fluidic sample containing a plurality of analytes, the method including:
a) loading the sample in a sample channel;
b) selectively transporting a first group of analytes, including at least some target analytes, through a first junction to a separation channel in accordance with a size of a first free transport region of the first junction, the first group being a subset of the plurality of analytes in the sample; and,
c) selectively transporting a second group of analytes to a waste channel through a second junction in accordance with a size of the free transport region of the second junction, the second group being a subset of the first group, so as to concentrate a number of the target analytes in the separation channel.

Typically the method includes applying a first electric potential between at least one first electrode in the sample channel and at least one second electrode in the waste channel so as to selectively transport analytes through the first and second junctions.

Typically the method includes applying a second electric potential between third and fourth electrodes in the separation channel so as to selectively transport analytes from the first group of analytes within the separation channel.

### BRIEF DESCRIPTION OF THE DRAWINGS

An example of the present invention will now be described with reference to the accompanying drawings, in which:—

FIG. 1 is a schematic diagram of a first example of a device for at least partially extracting and concentrating a target analyte from a fluidic sample containing a plurality of analytes;

FIG. 2 is a flow diagram of a first example of a method of at least partially extracting and concentrating a target analyte from a fluidic sample containing a plurality of analytes;

FIG. 3 is a flow diagram of a first example of a method of manufacturing a device for at least partially extracting and concentrating a target analyte from a fluidic sample containing a plurality of analytes;

FIG. 4 is a schematic diagram of a further example of a device for at least partially extracting and concentrating a target analyte from a fluidic sample containing a plurality of analytes;

FIG. 5 is a flow diagram of a further example of a method of at least partially extracting and concentrating a target analyte from a fluidic sample containing a plurality of analytes;

FIG. 6A is an image of experimental results showing negatively charged bovine serum albumin, labelled with fluorescamine, concentrates at the tip of a sample channel of an example device;

FIG. 6B is an image of experimental results showing fluorescein, a small anion similar to a target analyte, is electrokinetically transported from the sample chamber into the separation channel of an example device;

FIG. 6C is an image of experimental results demonstrating that thiocyanate ions were electrokinetically transported from the sample channel of an example device, through the second junction into the waste channel;

FIG. 7A is a graphical representation of the sample matrix ionic strength in different concentrations over time;

FIG. 7B is a graphical representation of the sample matrix viscosity varied using hydroxy propyl methyl cellulose (HPMC) in different concentrations;

FIG. 7C is a graphical representation of the ionic strength of the background electrolyte in the separation channel;

FIG. 8 is a graphical representation of electropherograms comparing an example of a device for at least partially extracting and concentrating a target analyte from a fluidic sample containing a plurality of analytes, with conventional pinched injection, under the same conditions;

FIGS. 9A and 9B are graphical representations of electropherograms which compare the results obtained using nanopores created with a current limit of 9 μA to those created with the optimized current limit of 5 μA; and,

FIG. 10 is a schematic diagram of a further example of a device for at least partially extracting and concentrating a target analyte from a fluidic sample containing a plurality of analytes.

### DETAILED DESCRIPTION OF THE PREFERRED EMBODIMENTS

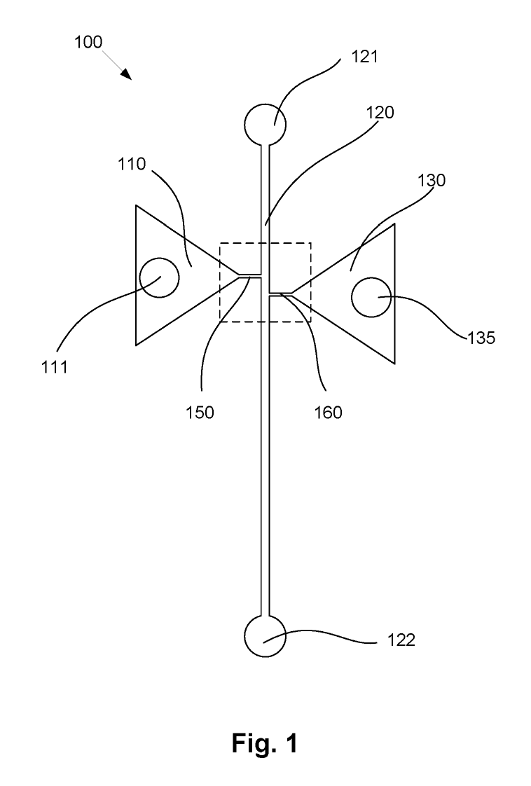

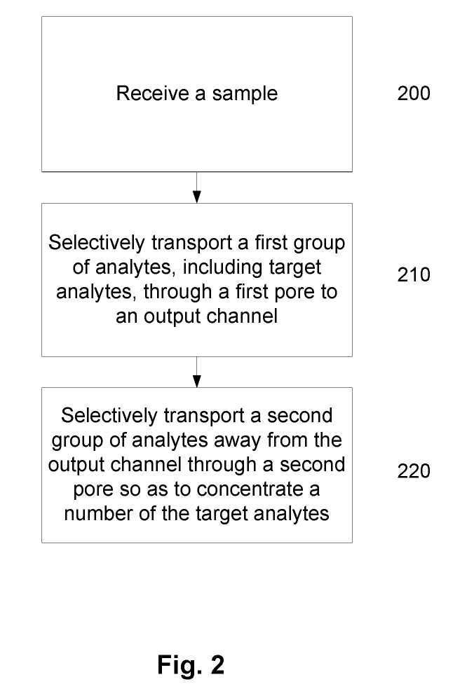

An example of a device for at least partially extracting and concentrating a target analyte from a fluidic sample containing a plurality of analytes will now be described with reference to FIGS. 1 and 2.

In this example, the device 100 includes a sample channel 110, a separation channel 120, and a waste channel 130. A first junction 150 is provided between the sample channel 110 and the separation channel 120, and a second junction 160 is provided between the separation channel 120 and the waste channel 130.

In use, a method of at least partially extracting and concentrating a target analyte from a fluidic sample containing a plurality of analytes includes, at step 200, loading the sample into the sample channel 110. At step 210, the method includes selectively transporting a first group of analytes through the first junction 150 from the sample channel 110 to the separation channel 120 in accordance with a size of a first free transport region of the first junction 150, the first group being a subset of the plurality of analytes in the sample and including at least some target analytes.

At step 220, the method further includes selectively transporting a second group of analytes from the separation channel 120, to the waste channel 130, through the second junction 160, in accordance with a size of a second free transport region of the second junction 160, the second group being a subset of the first group, so as to concentrate a number of the target analytes in the separation channel 120.

Thus, the device 100 and method facilitates the extraction and concentration of target analytes from a sample using a series of junctions with different sizes of free transport region. This arrangement is advantageous as it allows for the simultaneous extraction and concentration of a target analyte to be performed simply and easily, and in some examples, without the need for sample preparation, dilution, concentration, and the like. This is particularly beneficial as the device 100 may thus be used as a consumer analytical or diagnostic device, such as an on-chip device, or other sample in/answer out device, providing timely and accurate information to a user, consumer and/or clinician.

Moreover, the device 100 and/or method may be applied to any suitable target analyte, for example, a pharmaceutical or illicit drug, diagnostic marker, DNA, a protein, pathogen, other biomarker, or the like, according to the respective sizes of the first and second free transport regions, and this will be described in more detail below. Additionally, the device 100 and/or method may be usable with any suitable fluidic sample, such as whole blood, urine, saliva, milk, waste water, or the like.

A number of further features will now be described.

In one example, the first junction 150 includes at least one first junction channel extending between the sample channel and the separation channel, and wherein the size of the first free transport region is at least partially dependent upon at least one of an average pore size, and in particular a width (or height) or radius of the first channel and a degree of an electric double layer (EDL) overlap within the first channel. Similarly, in another example, the second junction 160 includes at least one second junction channel extending between the separation channel and the waste channel, and the size of the second free transport region is at least partially dependent upon at least one of an average pore size of the second junction 160 and a degree of an electric double layer overlap of the second junction 160.

For example, in the event the first and/or second junctions 150, 160 are substantially filled with an electrolyte, an EDL develops at the liquid-solid interface formed about the inner surface of the first and/or second junctions 150, 160. A thickness of the EDL is at least partially dependent upon the ionic strength of the electrolyte, such that decreasing the ionic strength increases the EDL thickness. In the event a sufficiently high electric field is provided across the first and/or second junctions 150, 160 for electrophoretic forces to overcome the electrostatic attraction forces from the inner surface, counter-ions, such as cations for negatively charged surfaces or anions for positively charged surfaces, carry current while co-ions are typically excluded. Thus, selective transport in the first junction 150 and/or second junction 160 is in accordance with a size and/or charge of each of the analytes in the first group and/or second group, respectively. It should also be noted that neutral analytes can also be transported using a charged “carrier”, such as a micelle. In this regard, this mechanism relies on the micelle carrying the molecule to the junction, and collapsing and releasing the neutral molecule as it goes through the second junction.

The first and/or second free transport region is also influenced by physical dimensions of the respective channels, such as a radius of the first channel and/or second channel, as only analytes with a radius smaller than the free transport region may be selectively transported. In this regard, the free transport region typically corresponds to the average pore size of the first channel and/or second channel, less the thickness of the EDL therein.

Whilst the first and/or the second junctions can include a single channel, the junctions can alternatively include a plurality of channels, for example provided in a bundle. The one or more channels could have a substantially circular cross sectional area, or alternatively could include an elongate cross sectional area, such as a single channel that has a limited height, such as 10 nm to thereby control the free transport size, whilst being substantially wider, such as 2 mm, allowing multiple analytes to be transported therethrough. The use of a wide channel or multiple channels, allows the free transport size to be controlled, whilst providing a high bandwidth, thereby increasing throughput. The junction(s) can also include a hydrogel and/or a membrane to further control transport of analytes. In one example, the membrane includes a hydrophilic polycarbonate track etch (PCTE) membrane, such as those available for purchase from Sterlitech Co. (Washington, USA).

The device 100 typically includes at least one first electrode 111 in the sample channel 110, and at least one second electrode 135 in the waste channel 130, to thereby apply a first electric potential across the first and second junctions 150 and 160, respectively. In this regard, the first and second electrodes 111, 135 enable the selective transport discussed above through the junctions 150, 160 leading to simultaneous extraction, concentration, and purification of the selected analytes. This step will be generally referred to as the injection step.

During the injection step, the electric potential may be applied to thereby sharpen the concentrated target analytes within a region of the separation channel 120. As will be discussed further below, applying the electric potential for a period exceeding a predetermined amount of time may lead to the onset of ion concentration polarisation (ICP) in which depletion and enrichment zones urge at least the target analytes in the separation channel 120 to the region. In some examples, the region is toward the second junction 160. Typically the region is a substantially narrow band transverse to the separation channel 120, which is therefore more beneficial in the event the target analytes subsequently undergo electrophoretic separation, as discussed in more detail below.

In particular, it will be appreciated that electrophoretic transport through the first and/or second junctions 150, 160 at least partially depends upon the direction and magnitude of the electrophoretic mobility of analytes in the junctions 150, 160 in comparison to respective electroosmotic flow (EOF). In this regard, surface charge density of the junctions 150, 160, ionic strength of the electrolyte within the junctions 150, 160, and the extent of EDL overlap determines the magnitude of the EOF, while surface charge, positive or negative, determines its direction.

After the injection step is completed the applied voltage can be switched to start optional electrophoretic separation. In this regard, the device 100 includes third and fourth electrodes 121, 122 in the separation channel to thereby apply a second electric potential along the separation channel so as to selectively transport analytes from the first group of analytes within the separation channel. Specifically, the target analytes within the separation channel 120 migrate along the separation channel 120 at a speed determined by one or more of their size, charge ratio and electrophoretic mobility. The separation channel can include a detector so that the target analytes are transported past the detector allowing a concentration of the target analytes within the sample to be detected, and this will be discussed in more detail below.

However, it will be appreciated that electrophoretic separation may not be required and the concentrated analytes in the separation channel could be used in other ways. For example, reagents could be added allowing a reaction to be performed. Alternatively, analytes could be extracted from the separation channel, allowing the analytes to be subsequently analysed remotely of the device.

In some examples, the sample, separation and waste channels 110, 120, 130, may be any suitable shape, size, and/or respective orientation. In this regard, the sample channel 110 and/or the waste channel 130 may be tapered toward the separation channel 120. More typically the sample channel and the waste channel are typically either substantially “V” or “U” shaped with two channel arms. In this example, double electrodes are typically provided, with a respective electrode proximate a first end of each arm, and the junction being provided proximate a second opposing end of each arm. This allows, for example, a higher transport rate of target analytes in a sample. It should be noted that, the “V” shaped design of the sample channel 110 limits the area in which first junction 150 may form between the sample channel 110 and separation channel 120, and hence a “U” shaped arrangement may be preferred.

In some examples, the first junction 150 and the second junction 160 are located on opposing sides of the separation channel 120, however this is not essential and any suitable location around the separation channel may be used. Typically, the first junction 150 and the second junction 160 are offset along a length of the separation channel. This may be particularly beneficial in band narrowing of the target analyte in the separation channel 120, for example, which in turn may improve electrophoretic separation. In particular, an offset allows ion transport through the first junction 150 and minimizes interference from the ion concentration polarisation (ICP) created by the second junction 160, as discussed further below.

In a further example, the selective transport in the second junction 160 is at least partially controlled in accordance with electrophoretic mobility, EOF, EDL overlap, and/or ion concentration polarization (ICP), and this will be discussed further below.

In some examples, at least one of the sample channel 110, separation channel 120 and waste channel 130 are substantially filled with an electrolyte solution. In this regard, the type and concentration of electrolyte solution will be determined based upon the nature of the target analyte. In some examples, the electrolyte solution in one or more of the channels may be the same during formation and use of the first and/or second junctions 150, 160, however this is not essential and in other examples, the electrolyte solutions may be different during formation and use.

In the above example, a single second junction and waste channel is provided. However, this is not essential, and the device can include a number of second junctions spaced apart along the separation channel, and wherein each second junction is for removing respective analytes thereby allowing a number of different target analytes to be extracted and concentrated within the separation channel. In this example, analytes are progressively removed from the separation channel, allowing for multiplexing to more easily target multiple candidate analytes.

In another example, an apparatus can be formed from a number of devices arranged in sequence. In this example, the apparatus can be used for extracting and concentrating a number of target analytes from a fluidic sample, with each device being adapted to extract and concentrate a respective analyte and wherein the waste channel of an upstream device is in fluid communication with, or part of a sample channel of a downstream device, so that analytes of interest are successively extracted and concentrated by each device in turn.

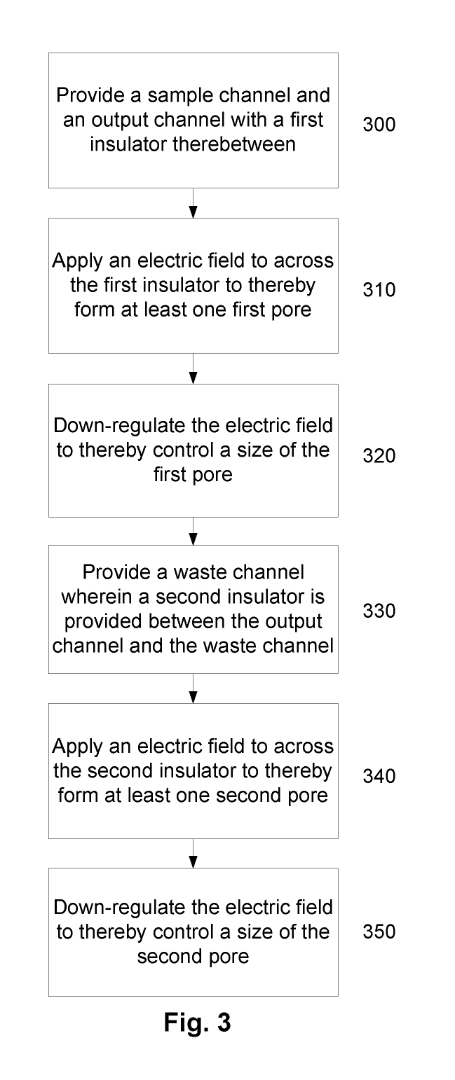

An example of a method of manufacturing a device for at least partially extracting and concentrating a target analyte from a fluidic sample containing a plurality of analytes will now be described with reference to FIG. 3.

In this example, the method includes at step 300 providing a sample channel and a separation channel with a first insulator therebetween. This may be achieved in any suitable manner, and in one example includes providing the sample channel and output channel on a chip, such as a chip substantially composed of one or more of polydimethylsiloxane (PDMS), glass, toner, or the like. In this regard, the first insulator may be substantially composed of one or more of PDMS, glass, toner, or the like.

In some examples, the sample channel and/or separation channel is substantially filled with an electrolyte solution, such as 1 mM disodium hydrogen phosphate, 10 mM phosphate buffer, 50 mM potassium chloride, or any other suitable electrolyte solution and/or concentration thereof as will be appreciated by persons skilled in the art.

In addition, the sample and/or separation channel may be provided in any suitable shape and orientation. In one particular example, the sample channel is provided as a compartment, which is tapered toward the separation channel, for example, as a substantially “V” shaped compartment. In addition, the sample channel may be provided as a “U” shaped compartment, wherein the base is directed toward the separation channel. However, in other examples, the channels may be any suitable shape, size, or orientation.

Additionally or alternatively, the sample and/or separation channel may be provided at any suitable proximity with respect to each other. For example, a distance between the sample channel and the separation channel is typically greater than or equal to about 10 μm, more typically about 50 μm and most typically less than or equal to about 1000 μm. As will be appreciated, the distance between the channels may be variable according to the relative shapes and orientations of the channels and thus the distances stated above are suggested only as examples.

At step 310, the method includes applying an electric field across the first insulator, where the electric field exceeds a dielectric strength of the first insulator, to thereby form at least one first junction. In this regard, the first junction extends between the sample channel and the separation channel. This may be achieved in any suitable manner, and in one example includes positioning an electrode in each of the sample and separation channel and applying an electric potential between the electrodes using a power supply with pre-set current limit, as will be discussed later.

In this regard, the applied electric potential is selected in order to exceed the dielectric strength of the first insulator, and therefore is typically selected based at least partially upon the dielectric strength and the distance between the channels. For example, an electric potential of 2.2 kV may be applied across a PDMS insulator (dielectric strength 21 V·μm⁻¹) which separates the sample channel and the output channel by about 100 μm.

At step 320, the method includes down-regulating or stopping the electric field following formation of the at least one first junction, to thereby at least partially control the pore size of the first junction. This may be achieved in any suitable manner, and in one example includes monitoring a return current from the power supply. In this respect, prior to the formation of the first junction, no current will be detected when applying the electric potential, however over time as first channel(s) form, the detected current will gradually increase in accordance with a cross-sectional area, also referred to as a pore size, of the channels in the first junction(s). Thus, maintaining the return current below a predetermined threshold by down-regulating or stopping the electric potential as the current reaches the threshold, allows at least partial control over the average pore size of the first junction. In this respect, whilst any suitable electric potential which exceeds the dielectric strength may be selected at step 310, typically an electric potential which is just above the dielectric breakdown allows better control as the rise in current is slower than with higher electric fields, giving time for the power supply to adjust without causing unwanted pore size widening.

Thus, the predetermined threshold of the return current will typically be determined in accordance with the desired application. For example, if using a power supply that down-regulates the applied voltage in response to return current and a 1 mM disodium hydrogen phosphate, in applying an electric potential of 2.2 kV across a PDMS insulator (dielectric strength 21 V·μm⁻¹) which separates the sample channel and the separation channel by about 100 μm, a threshold of 5 μA will typically yield a pore size of first junction channels which prevents, for example, red blood cells (6-8 μm in size) from transporting from the sample to the separation channel while allowing the transport of fluorescamine labelled bovine serum albumin (BSA). A threshold of 3 μA will typically result in first junction allowing transport of anionic 5-(and-6)-carboxynaphthofluorescein (CNF), but not BSA. A threshold of 2 μA results in a first junction which allows transportation of cationic dye rhodamine 6 G (R6G) and restricts CNF. A threshold of 1 μA results in a first junction which restricts transport of BSA, CNF and R6G, however allows transport of small ions, iron (III) and thiocyanate.

Accordingly, the predetermined current threshold influences the resultant average pore size of the first junction, thereby influencing selective transport through the first junction, as discussed in relation to any one of the examples described herein, and hence may be utilised for the extraction and concentration of target analytes. It should be noted that currents outlined above are particular to one example scenario and that different current limits may apply in different circumstances. For example, different results may be obtained if the power supply is changed, so a power supply that down-regulates the voltage as compared to one that stops the voltage may provide a different outcome. Also, the current will be different according to the electrolyte type and ionic strength.

At step 330, the method includes providing a waste channel, where a second insulator is provided between the separation channel and the waste channel. This may be achieved in any suitable manner, such as providing the waste channel on the chip with the separation channel and sample channel, as mentioned previously. Similarly, the chip and/or second insulator may be composed of any suitable material such as described above with respect to the first insulator. In this regard, the first and second insulator may be formed of the same material or different materials.

Also, as discussed above with relation to the sample channel, the waste channel may be arranged in any suitable shape, size, and/or orientation. In one example, the waste channel is tapered toward the separation channel, and typically the waste channel is a substantially “V” shaped compartment. In addition, the waste channel may be provided as a “U” shaped compartment, wherein the base is directed toward the separation channel. In a further example, the sample channel and the waste channel are provided on opposing sides of the separation channel, and in this regard, optionally the sample channel and the waste channel may be offset on the opposing sides.

In some examples, the waste channel is substantially filled with an electrolyte solution, such as 1 mM disodium hydrogen phosphate, 10 mM phosphate buffer, 50 mM potassium chloride, or any other suitable electrolyte solution and/or concentration thereof as will be appreciated by persons skilled in the art.

At step 340, the method includes applying an electric field across the second insulator, wherein the electric field exceeds a dielectric strength of the second insulator, to thereby form at least one second junction. This may be performed in any suitable manner, including any of the examples discussed above in relation to the application of an electric field across the first insulator.

The method further includes down-regulating or stopping the electric field, at step 350, following formation of the at least one second junction, to thereby at least partially control pore size of the second junction channels. This step may also be performed in any suitable manner, such as any of the examples described above in relation to the first junction. In some examples, it may be desirable for the relative sizes of the first and second junction channels to be different, and therefore typically a predetermined return current threshold for down-regulating or stopping the electric field applied across the second junction, also referred to as the second predetermined current threshold, will be different to the current threshold applied in respect of the first junction, also referred to as the first predetermined current threshold. More typically, the second predetermined current threshold will be less than the first predetermined current threshold, such that the resultant average pore size of the second junction channels is less than the average pore size of the first junction channels. In any event, this will be described in more detail below.

Alternatively, the first and/or second junction may be formed using a membrane, and this may be achieved, for example, instead of utilising the dielectric breakdown of an insulator. In one particular example, the membrane includes a hydrophilic polycarbonate track etch (PCTE) membrane, which may be PVP-coated and having a lowest non-specific binding. Typically, the membrane thickness of this example is about 6 μm, which is particularly beneficial as it reduces the risk of problems with PDMS bonding. In this example, the junctions typically range in diameter from 10 nm to hundreds of nanometers. However, this particular type of membrane is not essential and in other examples any suitable membrane may be used.

Whilst the abovementioned method steps are described in a particular order, it will be appreciated that the steps may be performed in any suitable order. For example, formation of the second junction may precede formation of the first junction, provision of the sample, separation and waste channels may occur simultaneously, or in any order, and the like.

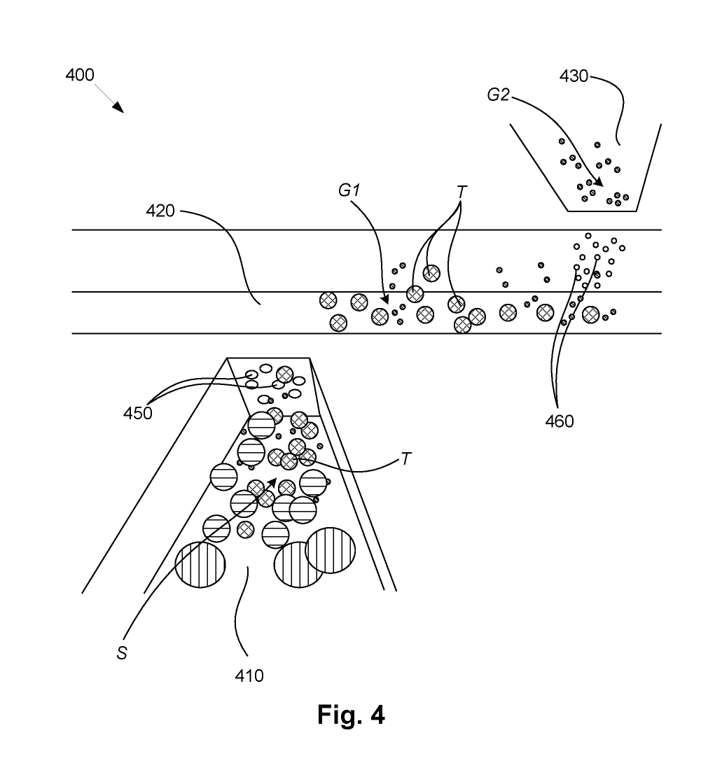

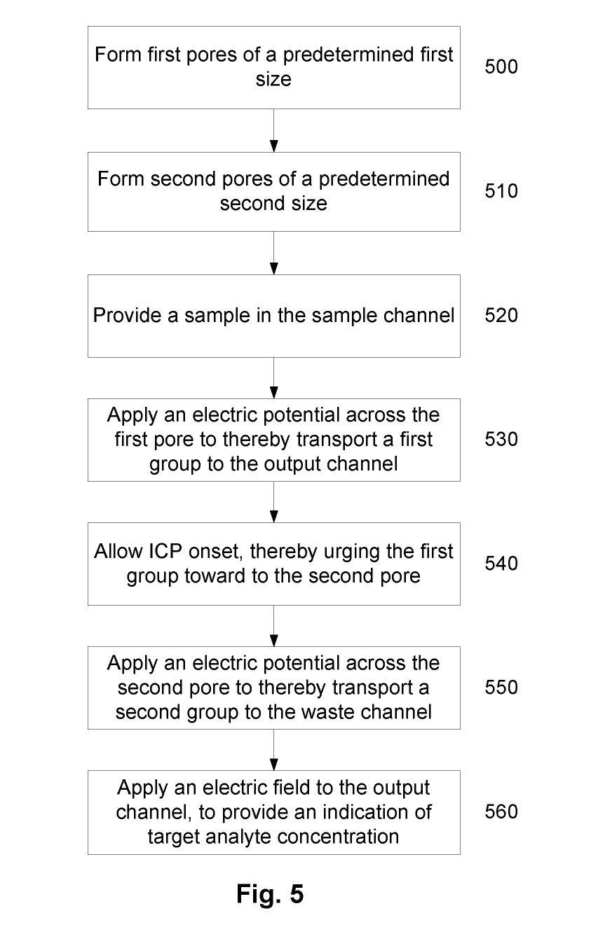

The device including the appropriate junctions created by the method mentioned above can be then used for at least partially extracting and concentrating a target analyte from a fluidic sample containing a plurality of analytes as shown in FIGS. 4 and 5. Features similar to those of the example described above have been assigned correspondingly similar reference numerals.

In this example, the device 400 includes a sample channel 410, separation channel 420 and waste channel 430. As shown, the separation channel 420 is in the middle, with the sample channel 410 and waste channel 430 provided on opposing sides of the separation channel 420. Additionally, the sample channel 410 is substantially perpendicular to, and tapered toward, the separation channel 420, with the waste channel 430 similarly arranged on the opposing side of the separation channel 420. A first insulator is provided between the sample channel 410 and the separation channel 420, and a second insulator is provided between the separation channel 420 and the waste channel 430. The sample, separation, and waste channels 410, 420, 430 in this example are also substantially filled with an electrolyte solution. In this regard, the type and concentration of electrolyte solution, and in addition, the type and geometry of insulator between the channels, may be determined based upon the nature of the target analyte.

At step 500, the method includes forming a first junction 450 of a predetermined first average pore size which extend between the sample channel 410 and the separation channel 420. This may be achieved in any suitable manner, such as described in the example above. The method further includes forming a second junction 460 of a predetermined second average pore size which extend between the separation channel 420 and the waste channel 420, and as described above, this may be performed in any suitable manner, as discussed herein. In this example, the average pore size of the first junction 450 is larger than the average pore size of the second junction 460, and this will be discussed in more detail below.

The first and second predetermined average pore size of the junctions will typically be dependent upon the target analyte, and in some examples, the nature of the fluidic sample. In this regard, typically the free transport region of the first junction will exceed a size of the target analyte while the free transport region of the second junction will be comparable or less than the size of the target analyte. Additionally, it will be appreciated that the first and second junctions may be formed at any suitable time, such as immediately prior to use, during manufacturing, or the like.

At step 520, the method includes loading the sample S in the sample channel 410. This may be achieved in any suitable manner, and typically involves a subject and/or clinician transferring the fluidic sample, such as blood, urine, saliva, or the like, into the sample channel 410. As shown in FIG. 4, the sample S in this example includes a plurality of analytes of differing sizes and charge.

An electric potential is applied, at step 530, across the first and second junctions 450, 460 in order to simultaneously transport a first group of analytes G1 from the sample channel 410 to the separation channel 420, concentrate them, and transport unwanted small ions, referred to as second group G2, from the separation channel 420 into the waste channel 430 through the second junction 460. Selective transport of the first group G1 is achieved based upon a number of factors, as described above, including the size of the first junction 450, respective charges of the first group of analytes G1, electrolyte concentration, EDL thickness, a free transport region of the first junctions 450, EOF magnitude and direction, and the like. The electric potential across the second junction 460 selectively transport a second group G2 from the separation channel 420 to the waste channel 430. Selective transport of the second group G2 is achieved based upon a number of factors, as described above, including the size of the second junction 460, respective charges of the second group of analytes G2, electrolyte concentration, EDL thickness, a free transport region of the second junction 460, EOF magnitude and direction, and the like. In this particular example, the second group G2 includes smaller ions than the target analyte T, and thus selective transport through the second junction 460 effectively concentrates the target analyte T in the output channel 420.

At step 540, the injection step 530 is allowed to continue long enough after the ionic transport of the target analytes T has stopped, so that ion concentration polarisation (ICP) is created by the first junction 450, thereby its depletion zone on the anodic side of the first junction 450 pushes the first group G1 toward the second junction 460 which is opposite to the direction of the EOF created by the second junction 460. As will be appreciated, ICP corresponds to the formation of enrichment and depletion zones on cathodic and anodic sides, respectively. This may be achieved in any suitable manner, and in one example includes waiting a predetermined time while applying the electric potential across the first junction 450. In this regard, typically the predetermined time for the onset of ICP is longer than that required by the second junction 460 to develop ICP and depend upon the size of the first junction, ionic strength of the electrolyte solution, presence of EOF modifier, and net surface charge of the first junction.

Thus, following transport of the first group G1 into the separation channel 420, onset of ICP sharpens the first group G1 injected plug within the separation channel 420 into a narrower band, which is advantageous during electrophoretic separation. This may be achieved, in one example, by the depletion zone expanding slowly across the separation channel 420, on the anodic side of the first junction 450, pushing the injected group towards the second junction 460.

At step 550, the method includes applying an electric field to the separation channel 420, to ensure electrophoretic separation of the first group G1 where each component migrates along the separation channel 420 at a speed determined by its size/charge ratio also known as electrophoretic mobility and passes the detector to provide an indication of its concentration including the target analyte T concentration. This may be achieved in any suitable manner, and in this example is achieved using electrophoretic separation.

In any event, the above provides an example of the on-chip simultaneous extraction, concentration, and purification of a target analyte T from a sample, utilising a device, which may be tailored according to the properties of the target analyte T, and optionally, the properties of the fluidic sample. This can be achieved by the presence of two junctions 450, 460 that differ in their average pore size and work as a system.

### Example—Blood Ampicillin Concentration

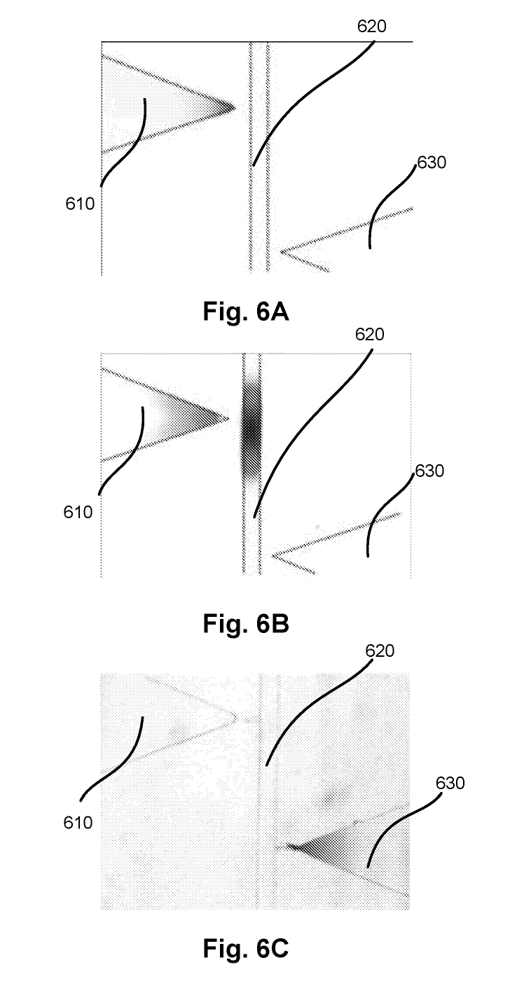

A further example of a device for at least partially extracting, concentrating, and purifying a target analyte from a fluidic sample containing a plurality of analytes will now be described with reference to experimental results and FIGS. 6A to 10. In this example, the sample includes whole blood, the target analyte is ampicillin, and a combination of two different pore size junctions in series is used to create molecular size and mobility traps (SMT) 1000 to selectively concentrate ampicillin from whole blood.

Ampicillin is an example of a first line antibiotic for treating sepsis and was selected as the target analyte for this work. Sepsis arises when the body's response to an infection damages its own tissues and organs, and is fatal for 30 to 50% of all patients. Hospitalizations for sepsis have more than doubled over the last 10 years, and in many countries, more people are hospitalized each year for sepsis than for heart attack. In the US, $14.6 billion was spent on hospitalizations for sepsis in 2008, and in Germany, the cost of a typical episode of sepsis has more than doubled over the last decade, from approximately 25,000 to 55,000 euros. In the treatment of sepsis, the selection of the dosage is particularly important, however difficult to determine, as a result of significant alterations in the pharmacokinetic behaviour of drugs due to hemodynamic alterations.

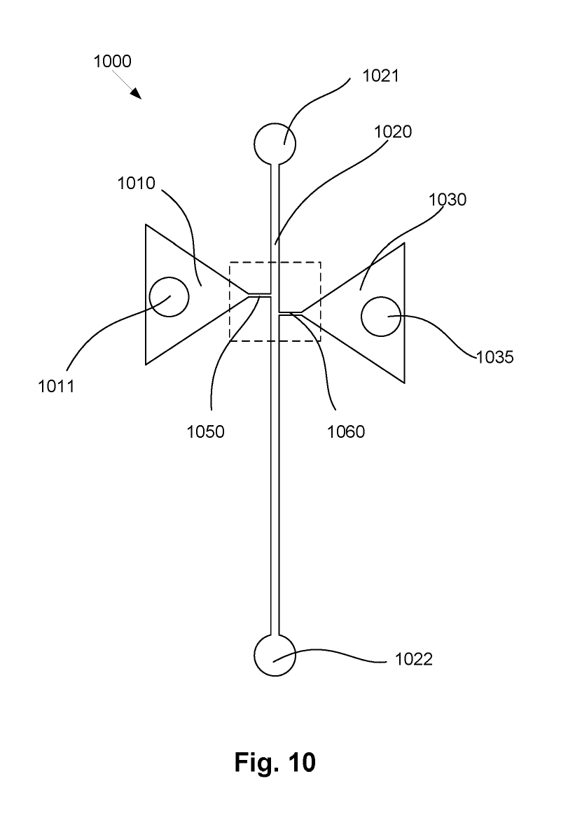

In this example, the device 1000 shown in FIG. 10 accepts about 40 μL of blood, which is typically equivalent to about one drop of blood, and provides the blood ampicillin concentration within about 3 minutes in order to inform dosing. Such a device 1000 is particularly advantageous as it aids care providers to make timely and informed decisions that in turn can save lives.

Ion transport through channels occurs naturally. When channels with a charged surface are filled with an electrolyte, an electric double layer (EDL) develops at the solid-liquid interface. For negatively charged surfaces, the Stern or immobile layer of cations forms closer to the surface and a diffuse layer of both anions and cations in which ions have some mobility forms further away. Neutral surfaces will not develop an EDL. The thickness of the EDL increases by decreasing the electrolyte ionic strength in the channel. When a high enough electric field is applied across the channel, the electrophoretic forces overcome the electrostatic attraction forces from the surface and the cations in the Stern layer will migrate towards the cathode, creating a cathodic or normal electroosmotic flow (EOF).

In channels, the EDL thickness is comparable to the channel dimensions, giving rise to two ion transport scenarios. If the channel dimension approaches twice the thickness of the EDL, EDL overlap leads to permselectivity of the pore, favouring the transport of counter ions in the EDL (cations in the case of a negatively charged surface) while excluding co-ions. The size of the channel is also important, as only counter-ions small enough to migrate through the EDL are transported. Conversely, when the EDL does not overlap, two regions can be distinguished in the channel: an electrostatic interaction region near the solid-liquid interface which surrounds a free transport region in the centre. The size of the counter-ions becomes important, as the physical size of this free transport region may be sufficient to permit small ions but hinder the transport of large biomolecules due to their size.

Electrophoretic transport through the nanopores also depends on the direction and magnitude of the electrophoretic mobility of the ion in comparison to that of the EOF. The surface charge density, ionic strength inside the channel, and the extent of EDL overlap determines the magnitude of the EOF while the surface charge, positive or negative, determines its direction. Consideration of these factors allows tuning of the permeability of a junction to selectively transport ions within a size and mobility range. Controlled selective ion transport is possible using junctions produced by dielectric breakdown, which provides a simple and cost effective technique for creating a junction between two microfluidic channels.

In this example, the device 1000 includes the “V” shaped sample and waste channels 1010, 1030, each having respective double electrodes 1011, 1035 positioned at a first end of the arms of the “V”, with junctions 1050, 1060 being formed at the second end of the arms by dielectric breakdown, a normally chaotic process, which was controlled by setting a current limit to down-regulate or stop the applied voltage once a current threshold, and resulting pore size, was achieved. In particular, we used two sequential junctions 1050, 1060 to form a size and mobility trap (SMT) 1000 to extract, concentrate, and purify target analytes. This allows size-selective electroextraction of small pharmaceuticals such as ampicillin through a first junction 1050 with larger pores followed by its concentration and desalting using differences in electrophoretic mobility and ion concentration polarization (ICP) created at the second junction 1060 containing smaller pores.

The device 1000 of this example, also referred to as the SMT 1000, was an irreversibly sealed PDMS/glass device with a 100 μm PDMS insulator between the tip of the “V” shaped sample and waste channels 1010, 1030 and a 50 μm wide separation channel 1020, through which the junctions 1050, 1060 were formed. In this regard, PDMS prepolymer and curing agent were purchased from an organisation trading under the trading name DowCorning (Sylgard 184). Fluorescein for fluorescent tracking was purchased from Sigma-Aldrich Co.

To cure the PDMS prepolymer, a mixture of 10:1 silicone elastomer and curing agent was poured on SU-8 templates prepared according to the manufacturer's procedure and placed at 60° C. for 1 hour. The templates used for the preparation of the microfluidic device 1000 had two V-channels 1010, 1030 with the dimension of 500 μm in width and 30 μm in height. The tips of the two channels 1010, 1030 were placed 100 μm on either side of the middle separation channel 1020 and 500 μm from each other along the length of the separation channel 1020. The separation channel 1020 was 50 μm in width and 30 μm in height. After curing, PDMS were detached from the templates. The microfluidic PDMS layer and the glass slide were irreversibly bonded after oxygen plasma treatment for 15 seconds.

The junctions 1050, 1060 from the sample and waste “V” shaped channels 1010, 1030 were positioned on opposite sides of the separation channel 1020 and offset by 500 μm. Prior to using the device, all channels 1010, 1020, 1030 were filled with 10 mM phosphate buffer, pH 11, and breakdown voltage of 2200 V was applied to the sample V-channel 1010 while keeping the separation channel 1020 grounded and setting the current limit to 5 μA.

The junction connecting the sample V-channel 1010 to the separation channel 1020 was created with a 5 μA current limit to create pores that block the transport of cells, plasma proteins and other macromolecules whilst permitting the transport of smaller ions (molecular weight<1000 Da) through the free transport region, not occupied by the EDL. During the injection step later, the high ionic strength on both sides of the junctions and the use of HPMC shields the surface charge, which delays the development of ICP and allows injection of small anions.

Next, the double electrodes with common input 1035 were connected to the waste “V” channel 1030 and the current limit was set to 1 μA. As relatively low voltage was used, no air bubble formation was observed. The channels 1010, 1020, 1030 were then cleaned and refilled with the experimental solutions. For the first use after the breakdown, reversed polarity was applied across the first junction at 1000 V till a current of 5 μA is reached then the proper voltage for the experiment is set.

After the formation of the junctions 1050, 1060, the sample was placed in the sample “V” channel 1010 and the separation channel 1020 filled with the background electrolyte (BGE, 100 mM phosphate buffer, pH 11.5, and 0.5% hydroxypropyl methyl cellulose (HPMC), unless stated otherwise) whilst the waste V-channel 1030 was filled with 10 mM phosphate buffer, pH 11.5. The PDMS surface is negatively charged at physiological and alkaline pH.

FIGS. 6A to 6C show the results of 3 different experiments using the abovementioned SMT. Ion depletion/enrichment zones were visualized by tracking fluorescence dye molecules that were added in the sample “V” channel 1010. Flow motions and ion transport images were obtained using an inverted fluorescence microscope (Ti-U, Nikon, Tokyo, Japan) integrated with Nikon CCD camera and fluorescence intensities were recorded using a PMT. A DC power supply was used to apply an electrical potential to each channel through an interface connected to platinum electrodes.

In this regard, the SMT includes the sample channel 610, separation channel 620, waste channel 630, and first and second junctions. These experimental results show negatively charged bovine serum albumin (Molecular weight˜66.5 kDa), labelled with fluorescamine, concentrates at the tip of the sample V-channel 610 in FIG. 6A, but is excluded from transport through the first junction into the separation channel 620 as its size exceeds the available free transport region.

Fluorescein, a small anion similar to the target analyte, is electrokinetically transported from the sample V-channel 610 into the separation channel 620, however is trapped in the separation channel 620 as shown in FIG. 6B. In this regard, the injected plug, or second group of analytes, concentrates in the separation channel 620 opposite to the first junction then shifts in position towards the second junction when ICP starts to develop at the first junction.

To visualise the transport of small inorganic anions for desalting, thiocyanate ions were electrokinetically transported from the separation channel 620 through the second junction into the waste V-channel 630, indicated by the formation of the red complex with acidic iron solution present in the waste V-channel 630 in FIG. 6C. Whilst electrophoretic transport of the iron ions into the thiocyanate reservoir is also expected, the red coloration was not observed in the sample V-channel 610 due to the high pH in this channel 610.

In this regard, the transport of fluorescein indicates no EDL overlap. The injection time was chosen to inject as much as possible then to use the ICP developed later to sharpen the injected plug into a narrow band ideal for electrophoretic separation. The depletion zone expands slowly across the separation channel, on the anodic side of the junction, and pushes the injected plug towards the second junction.

The junction between the waste V-channel 630 and the separation channel 620 was created with a 0.5 μA current limit to restrict the transport of small organic molecules but allows the transport of small inorganic ions to aid in desalting the sample while providing an optimum EOF for small molecule concentration. The trap was formed by balancing the analyte electrophoretic mobility with the EOF through the second junction as HPMC is not added to the waste V-channel 630 and lower ionic strength buffer was used to fill that channel 630. Lower ionic strength in the waste V-channel 630 allowed application of relatively high voltages without the risk of secondary breakdown and subsequent change in the average pore size of the second junction.

Together these two junctions create a size and mobility trap (SMT) in which molecules with a defined size/mobility range can be extracted and concentrated whilst smaller inorganic ions are transported to waste through the concentrating junction, enabling desalting of the concentrated analyte zone in the separation channel.

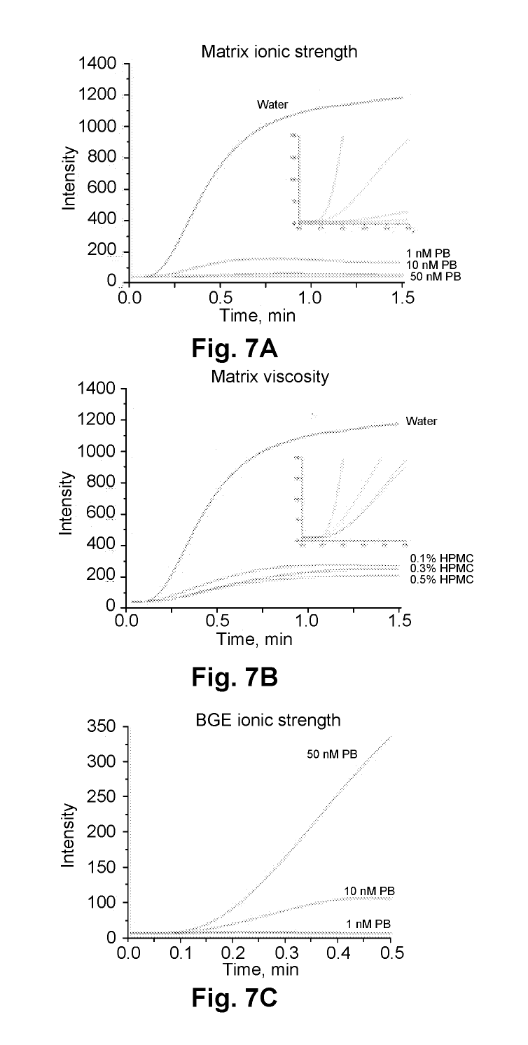

The matrix ionic strength and viscosity were found to affect the amount of transported ions, as shown in FIGS. 7A to 7C, which show the evolution of the of concentrated fluorescein zone as a function of injection time. In this regard, FIG. 7A shows the matrix ionic strength (0, 1, 10, and 50 mM phosphate buffer, pH 11.5) over time. FIG. 7B shows the matrix viscosity varied using hydroxy propyl methyl cellulose (HPMC) in different concentrations (0, 1, 3, and 0.5%). FIG. 7C relates to the ionic strength of the background electrolyte in the separation channel (2, 10, and 50 mM phosphate buffer, pH 11.5 containing 0.25% HPMC). 0.2 ppm fluorescein was used in all experiments, and the applied voltage was −100, −500, −100, and +300 V using a first separation channel electrode 1021, sample channel electrode 1010, second separation channel electrode 1022, and waste channel electrode 1030, respectively.

When dealing with samples of an invariable matrix, such as blood, the effect of the matrix ionic strength and viscosity on the amount of transported ions is not a problem, however an internal standard may be required for quantitative measurements from saliva or urine. In this respect, as the ionic strength and viscosity of the sample increases, ion transport quickly decreases. In order to avoid off-chip sample pretreatment or dilution, the BGE in the separation channel 620 was optimised so that enough analyte was injected. The Nernst-Planck equation describes the relation between the ion transport through a channel and the electric field gradient.

$$J ( x ) = - D \frac{\partial C ( X )}{\partial x} - \frac{zF}{RT} DC \frac{\partial ϕ ( x )}{dx} + {Cv}_{eo} ( x ) \qquad ( 1 )$$

where D, z, and C are the diffusion coefficient, charge of the permeation species, and concentration, respectively;
∂C(x)/∂x is the concentration gradient at distance x;
∂φ(x)/∂x is the potential gradient; and,
and νₑₒ (x) is the electroosmotic velocity.

The three terms on the right-hand side of equation (1) represent the contributions of diffusion, electromigration, and electroosmotic flow, respectively. Accordingly, using a high ionic strength BGE (100 mM) in the separation channel 620 increases the ion flux from the sample V-channel 610 even when dealing with high ionic strength samples like blood.

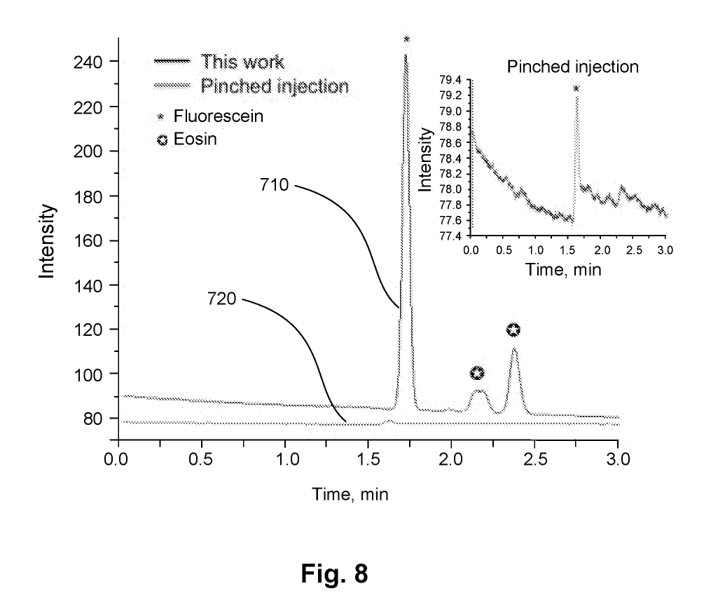

The concentration of the target analytes between the junctions is a combination of two phenomena. The EOF from the second, concentrating junction pushes the analyte back to the first, extraction junction, preventing spreading of the analyte zone across the separation channel. After some time, ICP is established in the first, extraction junction, with the forming depletion zone pushing the analyte towards the second junction. These two forces result in a narrow band, ideal for electrophoretic separation. The injection using the SMT resulted in a 100-fold concentration of fluorescein when compared with conventional cross geometry using pinched injection, and this is shown in FIG. 8.

In this regard, FIG. 8 shows electropherograms comparing SMT 710 with pinched injection 720 under the same conditions. Enhancement factors of 100-fold were achieved with SMT using a mixture of eosin and fluorescein in water (0.05 ppm each). Background electrolyte (BGE) in the separation channel was 100 mM phosphate buffer, pH 11.5, with 0.5% HPMC and in the waste V-channel was 10 mM phosphate buffer, pH 11.5. Applied voltages for SMT were −100, −300, −100, and +500 V for 60 seconds and for separation was −200, +100, +1500, and +100 V at the first separation channel electrode 1021, the sample V-channel electrode 1010, the second separation channel electrode 1022, and the waste V-channel electrode 1030, respectively. For pinched injection, all channels were filled with 100 mM phosphate buffer, pH 11.5, with 0.5% HPMC. Applied voltage for injection was −60, −240, −100, and +400 V at buffer, sample, buffer waste, and sample waste reservoirs, respectively, and separation voltage was similar to that of the SMT.

The SMT was applied for the analysis of a blood sample spiked with ampicillin, labelled with fluorescamine for fluorescence detection. Fluorescamine reacts with primary amines in their protonated form to produce a fluorescent compound within seconds whilst the excess reagent hydrolyses into a non-fluorescent product. The devices were fabricated and stored for up to 3 months. Immediately before use, the junctions were created by dielectric breakdown and the device was filled with the labelled sample and BGE.

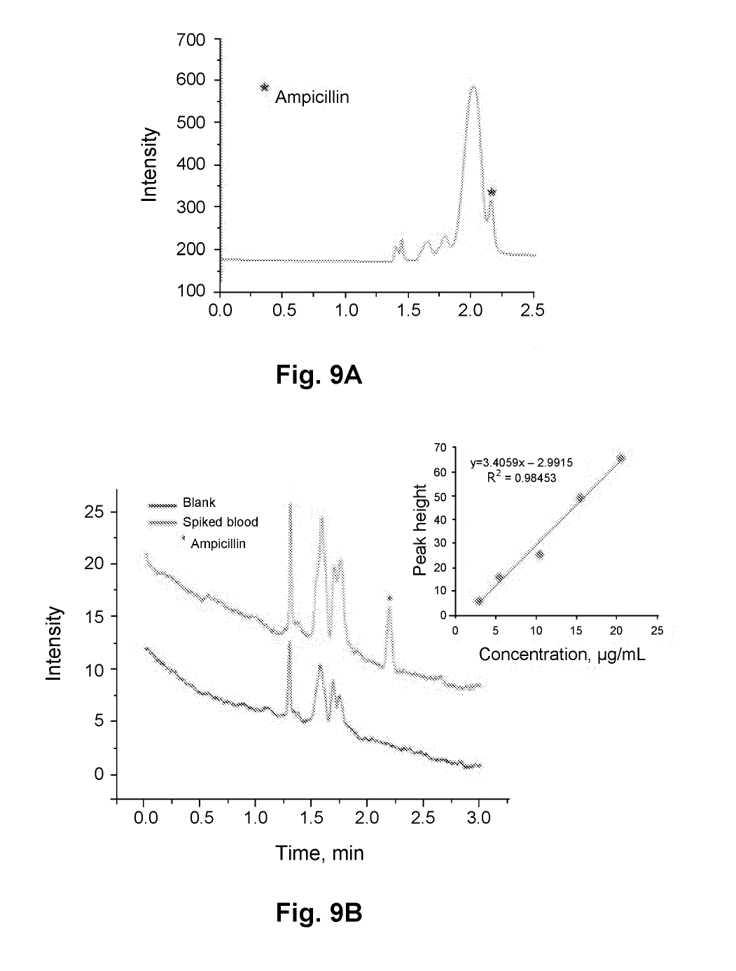

To demonstrate the importance of optimizing the pore size, the electropherograms in FIGS. 9A and 9B compare the results obtained for using filtration junction created with a current limit of 9 μA to that created with the optimized current limit of 5 μA. Although the wider pore size led to increased transport of ampicillin, they were less selective and large biomolecules interfered with the analysis, preventing quantification. When conducting the analysis with SMT devices where the optimum current limit of 5 μA was used, the fluorescamine labelled ampicillin was baseline separated from other fluorescent products, allowing quantification. The presented method exhibits a linear range of 2.5-20 μg/mL ampicillin, covering the recommended 10 μg/mL ampicillin blood level for treating neonatal sepsis, one of the major causes of death in new-borns.

In a further example, the device and/or method of any of the examples described herein may be applied to DNA as a target analyte. In one experiment, during injection the voltages applied to the first, second, third and fourth electrodes were −500 Volts, +200 Volts, −100 Volts and -100 Volts, respectively, for 200 seconds. During separation, the voltages applied to the first, second, third and fourth electrodes were +60 Volts, float, −60 Volts and +1500 Volts, respectively, for 200 seconds. The separation channel included a 100 mM phosphate buffer, pH 6.5 and 0.5% HPMC, and the waste channel included a 10 mM phosphate buffer, pH 6.5.

Changes in the sample matrix in this experiment were observed to effect the fluorescence intensity of the concentrated DNA in the separation channel, where intensity varied in accordance with DNA concentration. For example, a 50 nM DNA probe in a 10 mM phosphate buffer in the sample channel resulted in a higher concentration of DNA in the separation channel, than when a 100 mM phosphate buffer was included in the sample channel. In a further experimental example, blood was applied directly to the sample channel, and the resultant fluorescence of DNA in the separation channel was of similar magnitude to using a 100 mM phosphate buffer in the sample. A sample including a short DNA probe (substantially 20 base-pairs) in blood exhibited a variation in the resultant concentration of DNA in the separation channel, in accordance with concentration of the DNA probe. In particular, experimental results obtained using a 500 nM probe fluoresced at a higher intensity than a 50 nM probe, which in turn was higher than a 12.5 nM probe. In some experiments involving a 500 nM DNA probe, the present invention provided an 80-fold improvement in fluorescence, also referred to as an enhancement factor, over pinched injection.

In another example, the device and/or method of any of the examples described herein may be applied to one or more proteins as target analyte(s). In an experimental example, individual protein solutions were prepared including insulin (5.8 kDa, pI=5.3) at 5 mg/mL in 50 mM NaHCO3, β-lactoglobulin (18.4 kDa, pI=5.1) at 10 mg/mL in water, ovalbumin (44.3 kDa, pI=4.54, 4.9) at 20 mg/mL in water, and BSA (66.4 kDa, pI=4.7, 4.9) at 20 mg/mL in water. In individual vials, 20 μL of each protein solution (40 μL for insulin) were mixed with 120 μL (60 μL for insulin) of 100 mM NaHCO3, and then labelled with 60 μL (30 μL for insulin) freshly prepared fluorescamine (3 mg/mL in acetone). Equal volumes of the labelled protein and SDS sample buffer were mixed then heated to 100° C. using a heating block for 6 min. The SDS sample buffer included 100 mM Tris, 2% SDS, 4% Dithioerythritol (DTT), with pH adjusted to 6.8 with HCl. Each solution was diluted to a desired concentration with buffer. The buffer included 50 mM Tris-phosphate (pH 8.3), 30 mM SDS, 2.25% POP™. Sieving matrix, POP™, was added to the buffer (1:3) in the separation channel connecting the injection point to the buffer waste (BW). The sample waste (SW) V-channel was filled with 50 mM Tris-phosphate (pH 8.3).

In some examples according to any one of the embodiments described herein, it may be desirable to at least partially coat the device, and more typically to at least partially coat the sample channel. In this regard, coating can effect the concentration of resultant target analytes in the separation channel, which can be experimentally observed in accordance with fluorescence intensity of the resultant target analytes. In one example, the coating includes diethoxy (3-glycidyloxy propyl) methyl silane (DGPMS). In one experimental example, a higher fluorescence intensity of BSA in the separation channel (from a 200 μg/mL sample) was achieved using a DGPMS coated sample channel, when compared to a non-coated channel (where the breakdown voltage in both instances was the same, namely 4 μA). In the protein example described above, a higher experimental concentration of insulin, β-lactoglobulin, ovalbumin and BSA (and hence higher fluorescence intensity) was achieved in the separation channel with a coating of DGPMS:acetone (1:1).

Thus, the combination of two junctions with different pore size to achieve extraction, concentration, and desalting of small organic molecules from whole blood samples is shown in a single step. The method was applied for the analysis of the antibiotic ampicillin, a pharmaceutical compound with primary amine group available for labelling with fluorescamine. By adjusting the pore size of the junctions using the breakdown current limit, similar devices can be optimized for other analyte classes including biomarkers, proteins, or DNA. Fabrication of the junctions is fast, simple, and provides adequate control over pore size without adding to the cost of fabrication making it suitable for disposable devices. Furthermore, the electrokinetic device does not rely on pumps or valves, making operation simple.

Thus, the abovementioned examples of device and methods for at least partially extracting and concentrating a target analyte from a fluidic sample containing a plurality of analytes, which may be tailored according to the target analyte of interest, provide simple and timely results, may be easy to manufacture, and the like.

Additionally, the ability of the device to extract, concentrate, and purify a target analyte depends on a combination of physical characteristics such as pore size and material, as well as the electrolyte solution used, and therefore a particular device and solution combination could be used to target a particular analyte. Hence, this allows a cheap device to be produced, which can accurately and reliably measure specific target analytes, making this suitable for use as point-of-care device.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications, which become apparent to persons skilled in the art, should be considered to fall within the spirit and scope that the invention broadly appearing before described. Thus, for example, it will be appreciated that features from different examples above may be used interchangeably where appropriate.

What is claimed is:

1) A device for at least partially extracting and concentrating a target analyte from a fluidic sample containing a plurality of analytes, the device including:
a) a sample channel that receives the sample;
b) a separation channel;
c) a waste channel;
d) a first junction between the sample channel and the separation channel, wherein the first junction selectively transports a first group of analytes from the sample channel to the separation channel in accordance with a size of a first free transport region of the first junction, the first group including at least some target analytes and being a subset of the plurality of analytes in the sample; and,
e) a second junction between the separation channel and the waste channel, wherein the second junction selectively transports a second group of analytes from the separation channel to the waste channel in accordance with a size of a second free transport region of the second junction, the second group being a subset of the first group, so as to concentrate a number of the target analytes in the separation channel.

2) A device according to claim 1, wherein the first junction includes at least one first junction channel extending between the sample channel and the separation channel, and wherein the size of the first free transport region is at least partially dependent upon at least one of a size of the at least one first junction channel and a degree of an electric double layer overlap within the at least one first junction channel.

3) A device according to claim 1, wherein the second junction includes at least one second junction channel extending between the separation channel and the waste channel, and wherein the size of the second free transport region is at least partially dependent upon at least one of a size of the at least one second channel and a degree of an electric double layer overlap within the at least one second junction channel.

4) A device according to claim 1, wherein the selective transport in the first junction is in accordance with at least one of a charge and size of each of the analytes in the first group.

5) A device according to claim 1, wherein the selective transport in the second junction is in accordance with at least one of a charge and size of each of the analytes in the second group.

6) A device according to claim 1, wherein the device includes at least one first electrode in the sample channel, and at least one second electrode in the waste channel, to thereby apply a first electric potential across the first and second junctions so as to selectively transport analytes through the first and second junctions.

7) A device according to claim 6, wherein the first electric potential is applied to thereby sharpen the concentrated target analytes within a region of the separation channel.

8) A device according to claim 1, wherein the device includes third and fourth electrodes in the separation channel to thereby apply a second electric potential along the separation channel so as to selectively transport analytes from the first group of analytes within the separation channel.

9) A device according to claim 8, wherein the electric potential in the separation channel is used to cause target analytes to migrate at a speed based on at least one of a size, charge ratio, and electrophoretic mobility of analytes.

10) A device according to claim 1, wherein the device includes a detector that detects a concentration of the target analytes within the separation channel in use.

11) A device according to claim 1, wherein at least one of the first and the second junctions includes at least one of:
a) a plurality of channels;
b) a single channel having an elongate cross sectional area;
c) a hydrogel; and,
d) a membrane.

12) A device according to claim 1, wherein the first junction and the second junction are offset along a length of the separation channel.

13) A device according to claim 1, wherein at least one of the sample channel and the waste channel is at least one of:
a) tapered toward the separation channel;
b) substantially “V” shaped; and,
c) substantially “U” shaped.

14) A device according to claim 1, wherein the sample channel includes two sample channel arms, each arm having a respective first electrode proximate a first end and the first junction being provided proximate a second opposing end.

15) A device according to claim 1, wherein the waste channel includes two waste channel arms, each arm having a respective second electrode proximate a first end and the first junction being provided proximate a second opposing end.

16) A device according to claim 1, wherein the selective transport in the second junction is at least partially controlled in accordance with at least one of electrophoretic mobility, electroosmotic flow (EOF), and ion concentration polarization.

17) A device according to claim 1, wherein the device includes a number of second junctions spaced apart along the separation channel, and wherein each second junction is for removing respective analytes thereby allowing a number of different target analytes to be extracted and concentrated within the separation channel.

18) Apparatus for at least partially extracting and concentrating a number of target analytes from a fluidic sample containing a plurality of analytes, the apparatus including a number of devices according to claim 1, each device being adapted to extract and concentrate a respective analyte and wherein the waste channel of an upstream device is at least one of in fluid communication with and forms part of a sample channel of a downstream device.

19) A method of at least partially extracting and concentrating a target analyte from a fluidic sample containing a plurality of analytes, the method including:
a) loading the sample in a sample channel;
b) selectively transporting a first group of analytes, including at least some target analytes, through a first junction to a separation channel in accordance with a size of a first free transport region of the first junction, the first group being a subset of the plurality of analytes in the sample; and,
c) selectively transporting a second group of analytes to a waste channel through a second junction in accordance with a size of the free transport region of the second junction, the second group being a subset of the first group, so as to concentrate a number of the target analytes in the separation channel.

20) A method according to claim 19, wherein the method includes at least one of:
a) applying a first electric potential between at least one first electrode in the sample channel and at least one second electrode in the waste channel so as to selectively transport analytes through the first and second junctions; and
b) applying a second electric potential between third and fourth electrodes in the separation channel so as to selectively transport analytes from the first group of analytes within the separation channel.

21) (canceled)
